(19) United States
(12) Patent Application Publication
(10) Pub. No.: US 2025/0114724 A1
(43) Pub. Date: Apr. 10, 2025

(54) SYSTEM FOR PURIFICATION OF BIOACTIVE PROTEINS, AND RELATED METHODS

(71) Applicant: HP BIOENGINEERING, LLC, Lewes, DE (US)

(72) Inventors: Christopher Hlubb, Ellicott City, MD (US); Brandon Carter, Burley, ID (US)

(21) Appl. No.: 18/913,635

(22) Filed: Oct. 11, 2024

Related U.S. Application Data
(63) Continuation of application No. PCT/US2023/018165, filed on Apr. 11, 2023.
(60) Provisional application No. 63/329,606, filed on Apr. 11, 2022.

(51) Int. Cl. B01D 15/22; A23J 3/08; B01D 15/36; B01D 25/12; B01J 47/022; C07K 1/36; G01N 30/02; G01N 30/72; G01N 30/86

(52) U.S. Cl. CPC B01D 15/22; A23J 3/08; B01D 15/362; B01D 25/12; B01J 47/022; C07K 1/36; G01N 30/7233; G01N 30/8631; G01N 2030/027

(57) ABSTRACT

A system for purifying bioactive proteins in a fluid that includes at least one fractal subsystem and a filter press structured and arranged to support at least one fractal subsystem in a horizontal arrangement to provide a selectively adjustable bed depth, wherein each fractal subsystem is in fluid communication with every other fractal subsystem each fractal subsystem. The fractal subsystem may include a resin chamber and a pair of fractal packs, wherein the pair of fractal packs is disposed on opposing sides of the resin chamber. The resin chamber may include a frame, a plenum space through the frame for holding a resin, a pair of mesh screens for containing the resin within the plenum space, and a pair of feed material ports adapted to at least one of introduce a fluid into the plenum space or remove a fluid from the plenum space.

### CROSS-REFERENCE TO RELATED APPLICATION

This application is a continuation of International Application No. PCT/US2023/018165 filed Apr. 11, 2023, which claims the benefit of and priority to U.S. Provisional Patent Application No. 63/329,606, filed Apr. 11, 2022, the disclosure of which is hereby incorporated by reference in its entirety for all purposes.

### BACKGROUND OF THE INVENTION

The invention relates to systems and processes for purifying bioactive proteins and, more specifically, to a modular, rapid flow, ion exchange column; related systems; and related methods for purifying bioactive proteins, such as minor whey proteins contained, for example, in high-fat raw whole milk or colostrum-based feed material, and the like. Although the invention will be described in connection with purifying bioactive proteins, those of ordinary skill in the art can appreciate that the system and devices described herein may also be applied to processes for extracting metals (e.g., lithium and the like) from liquids or brine.

The isolation and purification of proteins (e.g., minor whey proteins and the like) contained in a liquid (e.g., high-fat milk, colostrum-based feed material, and the like) often requires ion exchange. However, conventional ion exchange chromatography systems are limited due to their design and function. For example, in addition to not being able to purify high-fat milk fees sources, conventional ion exchange chromatography systems, inter alia, are expensive; have a fixed height (or length); have a relatively large, non-modular footprint; suffer from low flow rates; lack uniform feed material flow across resins contained in the system; lack bidirectional flow; lack the capability to adjust a bed depth height (or length); and lack a uniform elution gradient flow.

### SUMMARY OF THE INVENTION

In a first aspect, the present invention relates to a fractal subsystem for use with an ion exchange system. In some embodiments, the fractal subsystem includes a resin chamber and a pair of fractal packs, wherein the pair of fractal packs is disposed on opposing sides of the resin chamber. In some implementations, the resin chamber may include a frame, a plenum space through the frame for holding a resin, a pair of mesh screens for containing the resin within the plenum space, and a pair of feed material ports adapted to at least one of introduce a fluid into the plenum space or remove a fluid from the plenum space.

In some variations, the mesh screens includes a 200 mesh, plain Dutch weave, have a nominal opening of 75 microns in the mesh, and/or have a geometric opening of 85 microns in the mesh.

In some applications, the fractal packs may include a frame, a plenum space through the frame, a fractal distributor configured within the plenum space, a fractal flow plate configured within the plenum space, and a feed material input port adapted to introduce a fluid into the plenum space. A weir may be formed within the plenum space in each of the upper corners of the frame.

In a second aspect, the present invention relates to a system for purifying bioactive proteins in a fluid. In some embodiments, the system includes at least one fractal subsystem and a filter press structured and arranged to support at least one fractal subsystem in a horizontal arrangement to provide a selectively adjustable bed depth, wherein each fractal subsystem is in fluid communication with every other fractal subsystem each fractal subsystem. In some embodiments, the fractal subsystem includes a resin chamber and a pair of fractal packs, wherein the pair of fractal packs is disposed on opposing sides of the resin chamber. In some implementations, the resin chamber may include a frame, a plenum space through the frame for holding a resin, a pair of mesh screens for containing the resin within the plenum space, and a pair of feed material ports adapted to at least one of introduce a fluid into the plenum space or remove a fluid from the plenum space.

In some variations, the mesh screens includes a 200 mesh, plain Dutch weave, have a nominal opening of 75 microns in the mesh, and/or have a geometric opening of 85 microns in the mesh.

In some applications, the fractal packs may include a frame, a plenum space through the frame, a fractal distributor configured within the plenum space, a fractal flow plate configured within the plenum space, and a feed material input port adapted to introduce a fluid into the plenum space. A weir may be formed within the plenum space in each of the upper corners of the frame.

In some implementations, the filter press may include a pair of vertical supports, a pair of horizontal supports fixedly attached to the vertical supports, wherein the horizontal supports are structured and arranged to support the fractal packs, and a filter press subsystem that is structured and arranged to apply a compressive force to the fractal packs. In some variations, the filter press subsystem may include a selectively movable rod, a follower disposed at a distal end of the movable rod, and a hydraulic press disposed at a proximal end of the movable rod.

Advantageously, the selectively adjustable bed depth may range between one (1) inch and eight (8) feet; the system is capable of bidirectional flow; the system produces laminar flow in the fluid; and/or the fluid may include a supply feed material with a fat content greater than 0.1 percent and less than five (5) percent.

Although the invention will be described in connection with proteins that include minor whey proteins and a liquid that includes high-fat raw whole milk or colostrum-based feed material, and the like, those of ordinary skill in the art can appreciate that the concepts described herein may also be applied to food, wastewater, water treatment, electronic devices, industrial catalysts, carbohydrate refinement, extraction of metals from liquid or brine, and active pharmaceutical ingredient manufacturing environments in which ion exchange may be required.

### BRIEF DESCRIPTION OF DRAWINGS

The accompanying figures, which are included as part of the present specification, illustrate the presently preferred embodiments and together with the general description given above and the detailed description of the preferred embodiments given below serve to explain and teach the principles described herein.

FIG. 1 shows an illustrative fractal subsystem according to some embodiments of the present invention;

FIG. 2A shows a first side perspective view of an ion exchange system including the fractal subsystem of FIG. 1, in accordance with some embodiments of the present invention; and

FIG. 2B shows a second side perspective view of an ion exchange system including the fractal subsystem of FIG. 1, in accordance with some embodiments of the present invention.

### DETAILED DESCRIPTION

### Process:

Although those of ordinary skill in the art can use the functions and principles taught in this disclosure for a number of applications, including, but not limited to, extracting metals (e.g., lithium and the like) from a liquid or brine, these functions and principles will be described illustratively in the context of purifying a fluid such as raw milk for human consumption, pharmaceutical API manufacturing, and so forth. Whereas conventional systems are limited to purifying supply feed material having a fat content of no more than 0.1 percent, advantageously, the present system and process are capable of processing highly-viscous supply feed materials with a fat content of up to about five (5) percent. Such high-fat content fluids would clog conventional systems.

In a first step, a high resolution of separation of undesirable proteins having a positive or a negative charge from the raw milk is desirable. Hence, before the raw milk becomes skim milk, the fluid (e.g., raw milk) may be diverted to an ion exchange system, described in greater detail below, to remove one or more (e.g., minor whey) charged proteins. Advantageously, the ion exchange system contains a (e.g., oppositely-charged chromatograph) resin that is adapted to separate proteins having different charges from the liquid.

Those skilled in the art are aware that the net surface charge of the liquid is a function of pH and, moreover, that a protein has no net charge at its isoelectric point (pI). Accordingly, if the pI is greater than the pH, the protein will bind to positively-charged ions in an anion exchanger, whereas if the pI is less than the pH, the protein will bind to negatively-charged ions in a cation exchanger. Once these charged proteins have been removed, the liquid may be returned to the conventional (e.g., dairy) purification process where it initially becomes skim milk.

In greater detail, the process includes four phases. First, the system columns are prepared at or for a desired pH. Next, the liquid may be introduced into the column and subject to the ion exchanger, such that the target molecules in the liquid bind to the ion exchanger. Unbound material may be washed out of the column, while the bound biomolecules are eluted such that they are gradually released from the ion exchanger due, for example, to a change in buffer composition. Finally, in a regeneration step, target molecules that remain bound to the ion exchanger may be removed. Even more detail on an example process if provided in the following paragraphs, as well as in U.S. Provisional Patent Application No. 63/486,437, which is incorporated by reference herein in its entirety.

Purification methods can reduce, minimize, or eliminate chemical treatment, enzymatic treatment, acid treatment, and/or heat treatment. Purification methods can reduce chemical treatment, enzymatic treatment, acid treatment, and/or heat treatment. Purification methods can minimize chemical treatment, enzymatic treatment, acid treatment, and/or heat treatment. Purification methods can eliminate chemical treatment, enzymatic treatment, acid treatment, and/or heat treatment. Purification methods can reduce, minimize, or eliminate chemical treatment. Purification methods can reduce, minimize, or eliminate enzymatic treatment. Purification methods can reduce, minimize, or eliminate acid treatment. Purification methods can reduce, minimize, or eliminate heat treatment. Purification methods can reduce each of a chemical treatment, enzymatic treatment, acid treatment, and heat treatment. Purification methods can minimize each of a chemical treatment, enzymatic treatment, acid treatment, and heat treatment. Purification methods can eliminate each of a chemical treatment, enzymatic treatment, acid treatment, and heat treatment. Purification methods can eliminate each of a chemical treatment, enzymatic treatment, and acid treatment. Purification methods can eliminate each of a chemical treatment, enzymatic treatment, and heat treatment.

In general, methods provided herein for the production of purified Lactoferrin do not include heat treatments typical in industrial purification processes (e.g., pasteurization) that can disrupt a native conformational state (e.g., denaturing) with reference to conformational states found in the natural milk source used for Lactoferrin purification. Typical heat treatments used in industrial purification processes can be about 63° C. or greater. Heat treatments can be 50° C. or greater, 51° C. or greater, 52° C. or greater, 53° C. or greater, 54° C. or greater, or 55° C. or greater. Heat treatments can be 70° C. or greater, 75° C. or greater, 80° C. or greater, 85° C. or greater, 90° C. or greater, 95° C. or greater, or 100° C. or greater. Purification methods can include performing processes at temperatures that reduce, minimize, or eliminate disrupting a native conformational state with reference to conformational states found in the natural milk source used for Lactoferrin purification relative to heat treatments typical in industrial purification processes. Purification methods can include performing processes at temperatures that reduce, minimize, or eliminate a reduction in Lactoferrin bioactivity with reference to bioactivity found in the natural milk source used for Lactoferrin purification relative to heat treatments typical in industrial purification processes. Purification methods can include receiving the natural milk source at refrigerated temperatures (e.g., at a temperature of less than 15° C., such as within a temperature between 2-15° C.). Purification methods described herein can include heat treatments of less than 50° C. For example, milk sources can be warmed during one or more purification steps, e.g., warmed to a temperature above 37° C., but not to exceed 55° C. Warming can include a temperature above 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., or 55° C. Warming can include a temperature above 37° C. and at a temperature of less than 55° C. Warming can include a temperature above 40° C. and at a temperature of less than 55° C. Warming can include a temperature above 45° C. and at a temperature of less than 55° C. Warming can include a temperature above 50° C. and at a temperature of less than 55° C. Warming can include a temperature above 37° C. and at a temperature of 50° C. or less, 51° C. or less, 52° C. or less, 53° C. or less, 54° C. or less, or 55° C. or less. Warming can include a temperature above 40° C. and at a temperature of 50° C. or less, 51° C. or less, 52° C. or less, 53° C. or less, 54° C. or less, or 55° C. or less. Warming can include a temperature above 45° C. and at a temperature of 50° C. or less, 51° C. or less, 52° C. or less, 53° C. or less, 54° C. or less, or 55° C. or less. Purification methods described herein can include heat treatments of 50° C. or less, 51° C. or less, 52° C. or less, 53° C. or less, 54° C. or less, or 55° C. or less. Purification methods described herein can include maintaining a temperature during purification below 50° C. or less, 51° C. or less, 52° C. or less, 53° C. or less, 54° C. or less, or 55° C. or less. Maintaining a temperature during purification can include maintaining a temperature throughout purification. Maintaining a temperature during purification can include maintaining a temperature in one or more individual steps of a purification process (e.g., chromatography, filtration, and/or drying steps). A maintained temperature can include varying temperatures (e.g., differing temperature ranges) specific to one or more individual steps of a purification process.

Purification methods can include acid treatments. Without wishing to be bound by theory, acid treatments can be used for removal of caseins, such as treating a natural milk source or derivative prior to Lactoferrin purification at a pH capable of causing caseins to become insoluble in solution. In general, acid treatments provided herein for the production of purified Lactoferrin do not include acids treatments that disrupt a native conformational state (e.g., denature Lactoferrin) and/or reduce Lactoferrin bioactivity with reference to conformational states or bioactivity, respectively, found in the natural milk source used for Lactoferrin purification. Purification methods can include acid treatments at a pH of 4.0 or greater. Purification methods can include acid treatments at a pH of 3.0 or greater.

Purification methods can include chromatography. Purification methods can include ion exchange chromatography. Methods of chromatography, such as ion exchange chromatography, are known to those of skill in the art. The purification methods described herein will generally include cation exchange chromatography. Purification methods may include ion exchange chromatography steps in addition to cation exchange chromatography, such as both cation and anion exchange chromatography, including in any order and/or separated be one or more addition purification processes. Resins and matrices for ion exchange chromatography are known in the art. For example, cation exchange resins include, but are not limited to, polymethacrylate and agarose matrices. Purification methods can include high-pressure liquid chromatography (HPLC).

Chromatography methods in general include one or more equilibration and/or regeneration steps. An illustrative non-limiting example of equilibration and regeneration steps includes rinsing with (1) reverse osmosis water; (2) chemically pure 1M NaCl; and (3) rinsing again with reverse osmosis water.

Chromatography methods in general include a loading step. In general, the volume loaded is based on a pre-determined binding capacity of a resin and an estimation of the native Lactoferrin content of a raw milk feed material.

Chromatography methods in general include one or more elution steps, e.g., elution of purified Lactoferrin off a resin/column in ion exchange chromatography. Methods of elution are known to those of skill in the art. Elution methods can include two or more elution steps. Without wishing to be bound by theory, multiple elution steps can be used to first elute off contaminants (e.g., any other product other than Lactoferrin) and then elute off the desired product (e.g., Lactoferrin).

Elution methods can include two or more elution steps at different salt concentrations. Without wishing to be bound by theory, one or more initial elution steps (e.g., elution steps prior to the elution step containing the desired purified lactoferrin) can be performed to remove undesired proteins and other contaminants.

Elution methods can include a first elution step between 0.2-0.7M of a chemically pure NaCl solution. In general, a first elution step between 0.2-0.7M of a chemically pure NaCl solution is performed to remove undesired proteins and other contaminants. Without wishing to be bound by theory, a first elution can be monitored for completion by UV-Vis spectrometry and/or colorimetric assays monitoring the presence of contaminants such as lactoperoxidase and other enzymes native to the raw milk feed material. For example, a first elution step between 0.2-0.7M of a chemically pure NaCl solution can be performed until a lack of protein eluting off a resin is detected by UV-Vis spectrometry.

Elution methods can include a first elution step of 0.2M chemically pure NaCl solution. Elution methods can include a first elution step of 0.25M chemically pure NaCl solution. Elution methods can include a first elution step of 0.30M chemically pure NaCl solution. Elution methods can include a first elution step of 0.35M chemically pure NaCl solution. Elution methods can include a first elution step of 0.40M chemically pure NaCl solution. Elution methods can include a first elution step of 0.45M chemically pure NaCl solution. Elution methods can include a first elution step of 0.50M chemically pure NaCl solution. Elution methods can include a first elution step of 0.55M chemically pure NaCl solution. Elution methods can include a first elution step of 0.6M chemically pure NaCl solution. Elution methods can include a first elution step of 0.65M chemically pure NaCl solution. Elution methods can include a first elution step of 0.7M chemically pure NaCl solution. Elution methods can include a first elution step of a chemically pure NaCl solution below 1M.

Elution methods can include a second elution step of 1M chemically pure NaCl solution. In general, an elution using 1M chemically pure NaCl solution will elute lactoferrin from the resin. Elution methods can include a second elution step of about a 1M chemically pure NaCl solution.

Elution methods can include a first elution step between 0.2-0.7M of a chemically pure NaCl solution and a second elution step of 1M chemically pure NaCl solution. Elution methods can include a first elution step of 0.2M chemically pure NaCl solution and a second elution step of 1M chemically pure NaCl solution. Elution methods can include a first elution step of 0.25M chemically pure NaCl solution and a second elution step of 1M chemically pure NaCl solution. Elution methods can include a first elution step of 0.3M chemically pure NaCl solution and a second elution step of 1M chemically pure NaCl solution. Elution methods can include a first elution step of 0.35M chemically pure NaCl solution and a second elution step of 1M chemically pure NaCl solution. Elution methods can include a first elution step of 0.40M chemically pure NaCl solution and a second elution step of 1M chemically pure NaCl solution. Elution methods can include a first elution step of 0.45M chemically pure NaCl solution and a second elution step of 1M chemically pure NaCl solution. Elution methods can include a first elution step of 0.50M chemically pure NaCl solution and a second elution step of 1M chemically pure NaCl solution. Elution methods can include a first elution step of 0.55M chemically pure NaCl solution and a second elution step of 1M chemically pure NaCl solution. Elution methods can include a first elution step of less than 1M chemically pure NaCl solution and a second elution step of 1M chemically pure NaCl solution.

Elution methods can include a first elution step between 0.25-0.7M of a chemically pure NaCl solution and a second elution step of about a 1M chemically pure NaCl solution. Elution methods can include a first elution step of 0.20M chemically pure NaCl solution and a second elution step of about a 1M chemically pure NaCl solution. Elution methods can include a first elution step of 0.25M chemically pure NaCl solution and a second elution step of about a 1M chemically pure NaCl solution. Elution methods can include a first elution step of 0.30M chemically pure NaCl solution and a second elution step of about a 1M chemically pure NaCl solution. Elution methods can include a first elution step of 0.35M chemically pure NaCl solution and a second elution step of about a 1M chemically pure NaCl solution. Elution methods can include a first elution step of 0.40M chemically pure NaCl solution and a second elution step of about a 1M chemically pure NaCl solution. Elution methods can include a first elution step of 0.45M chemically pure NaCl solution and a second elution step of about a 1M chemically pure NaCl solution. Elution methods can include a first elution step of 0.50M chemically pure NaCl solution and a second elution step of about a 1M chemically pure NaCl solution. Elution methods can include a first elution step of 0.55M chemically pure NaCl solution and a second elution step of about a 1M chemically pure NaCl solution. Elution methods can include a first elution step of 0.60M chemically pure NaCl solution and a second elution step of about a 1M chemically pure NaCl solution. Elution methods can include a first elution step of 0.65M chemically pure NaCl solution and a second elution step of about a 1M chemically pure NaCl solution. Elution methods can include a first elution step of 0.70M chemically pure NaCl solution and a second elution step of about a 1M chemically pure NaCl solution.

Elution methods can include two or more elution steps at different pH levels. elution gradient. Elution methods can include two or more elution steps at different salt concentrations, different pH levels, and combinations thereof. Elution methods can include an elution gradient. Elution methods can include an salt elution gradient. Elution methods can include a pH elution gradient. Elution methods can include both a salt and a pH elution gradient.

For each of the above ion exchange chromatography steps (e.g., equilibration regeneration, loading, and/or elution) one of skill in the art will recognize the appropriate fluid velocities, e.g., as dependent on the choice of resin, apparatus, raw milk feed material, etc.

Purification methods can include filtration. Methods of filtration are known to those of skill in the art. Purification methods can include microfiltration (typically referring to filtration using a membrane pore size of 0.1 to 10 μm). Microfiltration can include a membrane pore size of 1-10 μm. Microfiltration can include a membrane pore size of 10 μm. Microfiltration can include a membrane pore size of 1-10 μm. Microfiltration can include a membrane pore size of 0.1-1 μm. Microfiltration can include a membrane pore size of 0.1 μm. Microfiltration can include a membrane pore size of 1 μm. Microfiltration can include a membrane pore size of 0.1, 0.2, 0.3, 0.3, 0.5, 0.6, 0.7, 0.8, 0.9, and/or 1 μm. Microfiltration can include a ceramic filter.

Purification methods can include ultrafiltration (typically referring to filtration using a membrane pore size of 0.01 to 0.1 μm). Ultrafiltration systems can also be referred to by molecular weight cutoff sizes designed for sized-based separation between the permeate and retentate. Ultrafiltration systems can include 5-30 kDa systems. Ultrafiltration systems can include a 5 kDa system. Ultrafiltration systems can include a 10 kDa system. Ultrafiltration systems can include a 15 kDa system. Ultrafiltration systems can include a 20 kDa system. Ultrafiltration systems can include a 25 kDa system. Ultrafiltration systems can include a 30 kDa system.

Purification methods can include both microfiltration and ultrafiltration, including in any order and/or separated be one or more addition purification processes. Purification methods can include multiple microfiltration and/or ultrafiltration steps, including in any order and/or separated be one or more addition purification processes. As an illustrative non-limiting example, a first pre-filtering microfiltration step (e.g., with a 10 μm filter) can be used prior to ion exchange chromatography, a second microfiltration step (e.g., with a 0.1-1.4 μm filter) can be used subsequent to ion exchange chromatography, followed by an ultrafiltration step (e.g., with a 5-30 kDa).

Purification methods can include a combination of chromatography and filtration, including in any order and/or separated be one or more addition purification processes. Purification methods can include a combination of multiple chromatography and/or filtration steps, including in any order and/or separated be one or more addition purification processes. Purification methods can include a combination of microfiltration, ultrafiltration, and ion exchange chromatography, including in any order and/or separated be one or more addition purification processes. In an illustrative non-limiting example, purification methods can include ion exchange chromatography (including elution), followed by microfiltration and then by ultrafiltration. In another illustrative non-limiting example, purification methods can include microfiltration, followed by ion exchange chromatography (including elution), next followed by additional microfiltration, and then by ultrafiltration.

Purification methods can include separation of a raw milk product into milk product derivatives, such as separating a natural milk source into skim milk and cream. For example, a raw milk product can be separated into milk product derivatives prior to chromatography and/or filtration. Methods of separating a raw milk product into milk product derivatives are known to those of skill in the art including, but not limited to, cold-bowl separation.

Following purification of Lactoferrin, the purified product can be dried. In general, drying methods provided do not include treatments that disrupt a native conformational state (e.g., denature Lactoferrin) and/or reduce Lactoferrin bioactivity with reference to conformational states or bioactivity, respectively, found in the natural milk source used for Lactoferrin purification. Methods of drying Lactoferrin are known to those of skill in the art including, but not limited to, freeze-drying/lyophilization, fluid-bed drying, and/or low-temperature spray-drying.

In some embodiments, there can be an assessment of the purity. Natural milk sources, including bovine milk, typically contain several protein components in addition to Lactoferrin including, but not limited to lactoperoxidase, lysozyme, caseins, immunoglobulins, lactalbumin, and lactoglobulin. Natural milk sources can also contain other components, such as fat and endotoxin. In general, the purification methods provided herein reduce, minimize, or eliminate components other than Lactoferrin. Without wishing to be bound by theory, removal of one or more of the additional components can improve Lactoferrin bioactivity and/or improve safety.

Provided herein are Lactoferrin compositions having an increased percentage of Lactoferrin by mass relative to the ratio present in an unprocessed Lactoferrin-comprising milk product.

Lactoferrin percentage can be assessed by mass-spectrometry including, but not limited to, matrix assisted laser desorption ionization-time of flight (MALDI-TOF) mass-spectrometry and/or Linear Trap Quadropole Orbitrap Velos mass-spectrometry. In general, assessment by mass-spectrometry comprises quantifying an area under a peak corresponding to Lactoferrin and areas under a peak that do not correspond to Lactoferrin. In some instances, Lactoferrin can be associated with multiple peaks, such as ionization peaks corresponding to Lactoferrin. Peaks corresponding to Lactoferrin can include peaks corresponding to a full-length post-translationally modified (e.g., glycosylated) Lactoferrin. Peaks corresponding to full-length post-translationally modified Lactoferrin generally range from about 80,000-86,000 m/z. The exact peak for a full-length post-translationally modified can change, such as reflecting different glycosylation statuses. In some instances, to be inclusive, peaks having an m/z between 79000-90000 can be considered corresponding to Lactoferrin. Areas under a peak that do not correspond to Lactoferrin include all other peaks, with the potential exception of an ionization peak associated with Lactoferrin around 41,500 m/z (e.g., having an m/z between 41000-42000). Areas under a peak that do not correspond to Lactoferrin can include peaks having an m/z between 18000-80000 (other than peaks having an m/z between 41000-42000). A particular comparison can also be made between Lactoferrin and peaks corresponding to Lactoperoxidase (e.g., peaks having an m/z between 77000 and 78000). Areas under a peak that do not correspond to Lactoferrin can include peaks having an m/z between 18000-45000 (other than peaks having an m/z between 41000-42000). Linear Trap Quadropole Orbitrap Velos mass-spectrometry can also quantify the percentage of Lactoferrin relative to other components in a sample. For example, Quadropole Orbitrap Velos mass-spectrometry can quantify the percentage of Lactoferrin through determining the percentage of Peptide Spectrum Matches (PSMs).

Lactoferrin percentage can be assessed by liquid chromatography, such as high-performance liquid chromatography (HPLC). Assessment by liquid chromatography can include quantifying an area under a peak corresponding to Lactoferrin and areas under a peak that do not correspond to Lactoferrin.

For assessment involving quantification of peaks, one or more of the areas under a peak that do not correspond to Lactoferrin present in an unprocessed Lactoferrin-comprising milk product can be below a limit of detection for the purified Lactoferrin composition. In some instances, each area under a peak that does not correspond to Lactoferrin can be below a limit of detection.

Lactoferrin percentage can be assessed by an enzyme-linked immunosorbent assay (ELISA). In some instances, an ELISA can distinguish the percentage of Lactoferrin in a native protein conformation, such as by using an antibody that specifically binds the native protein conformation of Lactoferrin.

A specific component typically present in unprocessed milk sources to be reduced, minimized, or eliminated when purifying Lactoferrin is Lactoperoxidase. In addition, Lactoperoxidase is frequently present in detectable amounts in purified Lactoferrin compositions produced by typical industrial methods.

Provided herein are Lactoferrin compositions where at least at least 70% of the composition is purified Lactoferrin. Compositions include those where at least 75%, at least 80%, at least 85%, at least 90%, or at least 95% of the composition is purified Lactoferrin. Compositions include those where at least 75% of the composition is purified Lactoferrin. Compositions include those where at least 80% of the composition is purified Lactoferrin. Compositions include those where at least 85% of the composition is purified Lactoferrin. Compositions include those where at least 90% of the composition is purified Lactoferrin. Compositions include those where at least 95% of the composition is purified Lactoferrin. Compositions include those where at least about 100% of the composition is purified Lactoferrin. Compositions include those where at least 91%, at least 92%, at least 93%, at least 94%, at least 95%, at least 96%, at least 97%, at least 98%, or at least 99% of the composition is purified Lactoferrin. Compositions include those where at least 91% of the composition is purified Lactoferrin. Compositions include those where at least 92% of the composition is purified Lactoferrin. Compositions include those where at least 93% of the composition is purified Lactoferrin. Compositions include those where at least 94% of the composition is purified Lactoferrin. Compositions include those where at least 95% of the composition is purified Lactoferrin. Compositions include those where at least 96% of the composition is purified Lactoferrin. Compositions include those where at least 97% of the composition is purified Lactoferrin. Compositions include those where at least 98% of the composition is purified Lactoferrin. Compositions include those where at least 99% of the composition is purified Lactoferrin.

Provided herein are Lactoferrin compositions having an increased ratio of Lactoferrin:Lactoperoxidase relative to the ratio in an unprocessed Lactoferrin-comprising milk product. Ratios of Lactoferrin:Lactoperoxidase can be assessed according to methods known to those skilled in the art, such as the purity assessment methods described herein (e.g., mass-spectrometry, HPLC, and/or ELISA). For example, assessment can include quantifying an area or areas under a peak corresponding to Lactoferrin and an area under a peak corresponding to Lactoperoxidase.

Endotoxin present or potentially present in natural milk sources can be reduced, minimized, or eliminated. Without wishing to be bound by theory, removal of endotoxin can improve the safety of a purified Lactoferrin composition. Methods of assessing endotoxin levels are known to those skilled in the art.

Also provided for herein are methods of assessing purity of a purified Lactoferrin composition, such as using any of the purity assessment methods described herein.

### Fractal Subsystem:

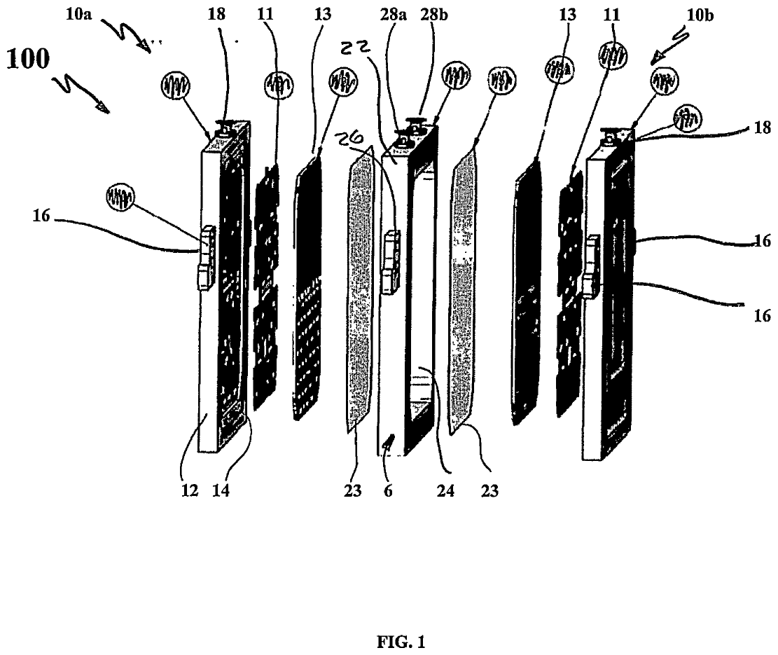

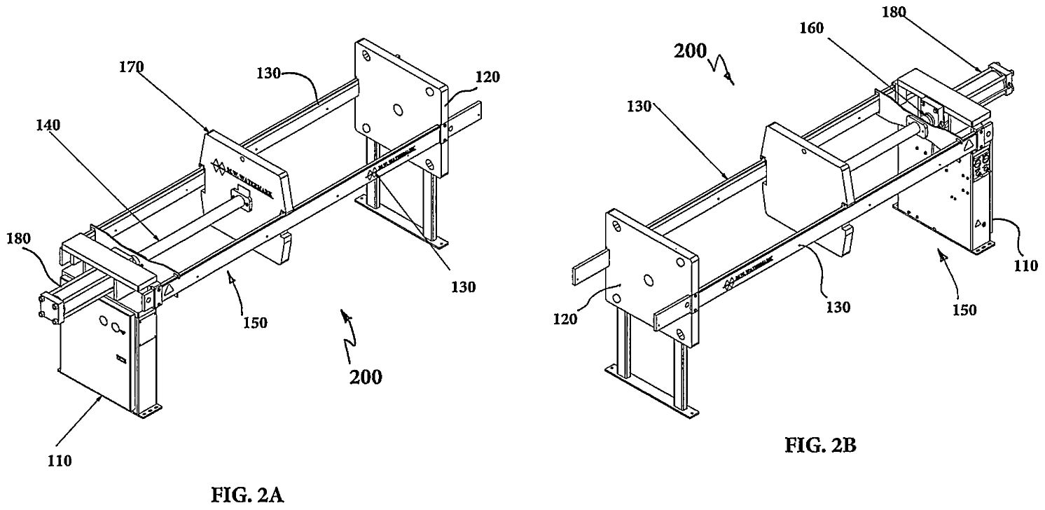

Referring to FIGS. 1, 2A, and 2B, an exemplary embodiment of a fractal subsystem 100 for use in connection with an ion exchange chromatography system 200 is shown. Unlike conventional mixing systems that include a columnar shape of a generally fixed height and having a substantially vertical orientation and that, furthermore, mix fluids using turbulence, a customizable fractal subsystem 100 uses scaling structures, whose shape can be arranged in any practical desired dimension (e.g., length) in a substantially horizontal orientation. More particularly, fractal subsystems 100 may be structured and arranged to achieve fluid scaling and mixing using a multiplicity of engineered channels that ensure scaling is complete before the components to be mixed contact one another. Advantageously, fractal scaling eliminates turbulence, large-scale process inhomogeneities, and/or side reactions.

Due to the myriad of applications to which it may be applied, the various components of the fractal subsystem 100 may be made from a variety of materials (e.g., metal, plastic, carbon fiber, and so forth). Beneficially, the ability to use a myriad of materials ensures that, for a discrete application, customized materials may be used such that the materials adhere to raw materials, cleaning-in-place chemicals, solvents, or elution buffer requirements. For use with food and pharmaceuticals, however, federal laws require that components of the fractal subsystem 100 be manufactured from FDA 3A-approved material. In one illustrative embodiment, components of the fractal subsystem 100 may be manufactured of polypropylene, such as Polystone® P Natural Homopolymer manufactured by Boedeker Plastics, Inc. of Shiner, Texas.

In some embodiments, the fractal subsystem 100 includes a (e.g., modular) resin chamber 20 disposed between a pair of fractal packs 10a, 10b. Such an arrangement in a single system 200 enables multiple fractal subsystems 100 and, correspondingly, multiple resin chambers 20. Advantageously, flow volumes may be selectively (e.g., evenly) split between multiple resin chambers 20 and flow velocities within each resin chamber 20 may be maintained equally across all of the resin beds. Having fractal packs 10a, 10b on opposing sides of the resin chamber 20 enables the flow direction to be reversed during processing, which provides greater resin utilization and reduces single point entry clogging. In some implementations, for the purpose of easier cleanability, a single fractal pack may be provided in combination with the resin chamber 20. The (e.g., modular) resin chamber 20 enables more efficient resin packing and removal. Indeed, enhanced resin packing and removal extends the useful life of the resin as backflushing of resin (e.g., in a clean-in-place operation) and the removal processes enhance cleaning success.

Advantageously, each of the fractal packs 10a, 10b may include a frame 12 that surrounds an inner plenum space 14. In some implementations, a fractal distributor 11 and a fractal flow plate 13 may be disposed within the inner plenum space 14, such that the fractal distributor 11 and a fractal flow plate 13 are releasably or removably attached to the frame 12. In some applications, the fractal distributor 11 may be structured and arranged to provide a channeled array that divides the incoming fluid evenly and in a laminar flow, further ensuring that the flow is provided evenly to the resin chamber 20. In some variations, the fractal flow plate 13 receives fluid from the fractal distributor 11 and, subsequently, supplies the fluid to the resin chamber 20. In some variations, the fractal distributor 11 may be a low volume distributor, which, advantageously, may create better peak resolution and less gradient mixing. More specifically, low systemic/component retention volume in the fractal distributor 11 allows for more precise solvent control and minimal mixing with prior step fluids. Furthermore, a low volume distributor provides a more even elution gradient contact with the resin and the supply feed material.

To prevent fluid from bypassing the resin, a (e.g., ⅜-inch) weir may be incorporated or integrated into the upper or top corners of the inner plenum space 14. Advantageously, providing a weir on the edge of the resin chambers 20 ensures 100 percent contact of the fluid with the resin of the raw material, which degree of fluid contact cannot be achieved using conventional columnar arrangements. For illustrative purposes only, the frame 12 and the inner plenum space 14 have a, generally, rectangular or rounded rectangular shape.

About an outer periphery of the frame 12, one or more (e.g., two) support arms 16 and one or more feed material ports 18 are fixedly attached to the frame 12. The support arms 16 are structured and arranged to enable users to handle the frames 12 and to support the frame 12 on a filter press 200, while the feed material ports 18 are structured and arranged to introduce a fluid into and/or removed a fluid from the fractal distributor 11 and/or the fractal flow plate 13.

In some implementations, the resin chamber 20 includes a frame 22 that surrounds an inner plenum space 24. For illustrative purposes only, the frame 22 and the inner plenum space 24 have a, generally, rectangular or rounded rectangular shape. In some implementations, one or more (e.g., two) mesh screens 23 may be disposed within the inner plenum space 24, such that the mesh screens 23 are releasably or removably attached to the frame 22. The size of the openings in the mesh screens 23 depend in large part on the type of resin being used. In one illustrative implementation, the mesh screens 23 may include a 200 mesh, plain Dutch weave (316SS) having an 85-micron geometric opening in the mesh and a 75-micron nominal opening in the mesh. Table I summarizes maximum, minimum, and nominal mesh opening dimensions.

Advantageously, the mesh pore size facilitates the passage of larger materials and more viscous products through the resin and screens 23. In operation, in one implementations, fluid may be introduced into the system 100 via a first feed material port 18 in a first fractal pack 10a and caused to flow through the resin chamber 20 and removed from the system 100 via a second feed material port 18 in a second fractal pack 10b. Advantageously, the system 100 is bi-directional; hence, fluid may be introduced into the system 100 via a second feed material port 18 in a second fractal pack 10b and caused to flow through the resin chamber 20 and removed from the system 100 via a first feed material port 18 in a first fractal pack 10a.

About an outer periphery of the frame 22, one or more support arms 26 and one or more resin ports 28a, 28b are fixedly attached to the frame 22. The support arms 26 may be structured and arranged to enable users to handle the frames 22 and to support the resin chamber 20 on the filter press 200, while the resin ports 28a, 28b may be structured and arranged to introduce resin into and remove resin from between the mesh screens 23.

TABLE I

| Property | Units | Minimum | Nominal | Maximum |
|---|---|---|---|---|
| Mesh diameter | microns | 0.1 | 75 | 300 |
| Flow pressure | psi | 0.1 | 20 | 40 |
| Flow rate | L/min | 0.1 | 125 | 10,000 |

### System:

Referring to FIGS. 2A and 2B, an exemplary filter press 200 for use with the fractal subsystem 100 is shown. Unlike conventional systems, the exemplary filter press 200 has an adjustable bed depth, whose depth depends on the number of fractal subsystems 100 introduced or incorporated into the filter press 200. An adjustable bed depth allows for high flow rates and low back pressure. The exemplary filter press 200 using a number of fractal subsystems 100 also enables directed flow, which minimizes channeling and, resultingly, provides greater resin utilization. Table I provides an exemplary nominal flow rate through the system 100. Those skilled in the art can appreciate that the achievable and desirable flow rates may be based on the design of the fractal packs 10a, 10b and the resin chamber 20.

Hence, due to the inherent modular abilities of this system 200, the resin chamber 20 may be designed to handle even faster velocities.

In some embodiments, the filter press 200 includes a pair of (e.g., vertical) supports 110, 120 that, inter alia, are structured and arranged to support a pair of horizontal or substantially horizontal supports or sidebars 130 that are structured and arranged to support one or more fluidically-coupled fractal subsystems 100. For example, in some variations, the sidebars 130 support the support arms 16, 26 disposed on opposing sides of the frames 12, 22 of the fractal packs 10a, 10b and resin chambers 20.

In some implementations, a filter press subsystem 150 may be disposed between and supported by the sidebars 130. In some variations, the filter press subsystem 150 may include a selectively movable rod 140 that is operatively coupled to a hydraulic press 160 at a proximal (e.g., fixed) end of the rod 140 and to a follower 170 at a distal (e.g., movable) end of the rod 140. A hydraulic cylinder 180 may also be disposed at the proximal end of the rod 140, such that operation of the hydraulic cylinder 180 causes the rod 140 to advance towards the distal end or to retract to the proximal end.

In operation, one or more fractal subsystems 100 may be disposed between the follower 170 and the distal vertical support 120. Advantageously, unlike conventional systems, the bed depth height (or length) is selectively adjustable and not fixed. Indeed, any practical number of fractal subsystems 100 may be disposed on the filter press 200 between the follower 170 and the distal vertical support 120. Thus, the column produced by the fractal subsystems 100 is mobile, modular, and oriented horizontally rather than vertically. Moreover, the bed depth of the column is selectively adjustable (e.g., between about 1 inch and eight feet). Advantageously, the fractal subsystem 100 and filter press 200 also permit bidirectional flow through the column.

Once a desired number of fractal subsystems 100 have been disposed on the filter press 200, the feed material portals 18 have been fluidically connected to a fluid source or a fluid drain, and the resin portals 28a, 28b have been fluidically connected to a resin source or a resin drain, operation of the hydraulic press 160 may cause the follower 170 to compress the fractal subsystem 100 towards the distal end of the rod 140. Advantageously, as the follower 170 compresses the fractal subsystem 100 and as a noncompressible liquid is introduced into the fractal subsystem 100 (e.g., via one or more of the feed material portals 18) the liquid is forced through the fractal distributor 11, the fractal flow plate 13, and the resin contained in the resin chamber 20. Advantageously, the combination of the filter press 200 and fractal subsystems 100 provides laminar flow, a uniform elution gradient flow, and uniform feed material flow across and through the resin disposed between the mesh screens 23 of the resin chamber 20. Since the fluid flow may, in some applications, be bidirectional, single outlet fouling of feed material against resin at the inlet may be reduced.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1. Purification of Lactoferrin

Lactoferrin was purified as illustrated in FIG. 1. Briefly, raw milk (untreated milk, e.g., not chemically, enzymatically, acid, or heat treated prior to purification of the Lactoferrin) was (1) diverted prior to entry into an industry standard milk processing workflow; (2) flowed through an ion exchange resin/column with effluent typically returned to the standard milk processing workflow and Lactoferrin-containing eluates collected; (3) collected eluates filtered; and (4) purified Lactoferrin processed. Notably, all other known Lactoferrin purification processes begin with a source of milk obtained after the industry standard milk processing workflow that typically includes heat pasteurization. In addition, a natural unprocessed milk product (e.g., raw milk) was used as a source material so that Lactoferrin included relevant post-translation modifications potentially involved in bioactivity, in contrast to recombinantly-produced Lactoferrin.

A chromatography column was used to isolate and purify bovine lactoferrin to retain its native post-translational modifications, glycosylation and bound iron. The column was loaded with the chosen cation exchange resin, either polymethacrylate or agarose matrix, and equilibrated and regenerated by rinsing with (1) reverse osmosis water; (2) chemically pure 1M NaCl; and (3) rinsed again with reverse osmosis water.

Raw, untreated, unprocessed bovine whole milk (less than 24 hrs. since milking) was obtained directly from a dairy transport or silo vessel before being separated, skimmed, heated and/or pasteurized. Lactoferrin was purified from both raw colostrum (RC) and raw whole milk (RM).

The milk was filtered for large particulates with a 10 μm filter and was warmed to above 37° C. and kept at a temperature below 63° C. (generally considered the starting temperature for pasteurization) and loaded into the chromatography column. The volume loaded was based on a pre-determined binding capacity and an estimation of the native Lactoferrin content of the raw milk feed material. During the loading process, all flow-through was returned to the pasteurizer balance tank to return the feed to the factory to the point prior to separation and pasteurization.

Once loading was completed, the resin was rinsed with reverse osmosis water to remove any remaining milk compounds which have not bound to the resin. A 1^{st} elution included washing of the resin with a chemically pure NaCl solution (0.2-0.7M) and was monitored and assessed for completion (i.e., a lack of protein eluting off the resin) by UV-Vis spectrometry and colorimetric assays monitoring the presence of contaminants such as lactoperoxidase and other enzymes native to the raw milk feed material. In particular, the 1^{st} NaCl was performed until lactoperoxidase, generally the major contaminant, was no longer present in the eluted fractions as assessed by peroxidase colorimetric assay using a colorimetric peroxidase substrate. The fraction of the 1^{st} elution was retained in an isolated vessel. The resin was then rinsed with reverse osmosis water. The 2^{nd} elution was performed with 1M NaCl to remove the isolated Lactoferrin. The fraction of the 2^{nd} elution was retained in an isolated vessel and then (1) filtered through a ceramic microfiltration filter (0.1-1.4 μm); and (2) concentrated using an ultrafiltration system (5-30 kDa).

The desalinated eluent was then freeze dried but may also proceed directly into a fluid bed dryer to be dried onto a pharmaceutical grade excipient. The purified lactoferrin produced according to the methods described herein are referred to as: Hyacinth lactoferrin (“Hyacinth”); Lactoferrin (solely without the accompanying words “Lab-grade” or “Commercially available supplement grade”; ODT-SC210; and API-E2. The various names may refer to purified lactoferrin produced according to variations of the methods described herein.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A fractal subsystem for use with an ion exchange system, the fractal subsystem comprising:
a resin chamber; and
a pair of fractal packs, wherein the pair of fractal packs is disposed on opposing sides of the resin chamber.

2. The fractal subsystem of claim 1, wherein the resin chamber comprises:
a frame;
a plenum space through the frame for holding a resin;
a pair of mesh screens for containing the resin within the plenum space; and
a pair of resin ports adapted to at least one of introduce a fluid into the plenum space or remove a fluid from the plenum space.

3. The fractal subsystem of claim 2, wherein the mesh screens comprise a 200 mesh, plain Dutch weave.

4. The fractal subsystem of claim 2, wherein the mesh screens have a nominal opening of 75 microns in the mesh.

5. The fractal subsystem of claim 2, wherein the mesh screens have a geometric opening of 85 microns in the mesh.

6. The fractal subsystem of claim 1, wherein each of the fractal packs comprises:
a frame;
a plenum space through the frame;
a fractal distributor configured within the plenum space;
a fractal flow plate configured within the plenum space; and
a feed material input port adapted to introduce a fluid into the plenum space.

7. The fractal subsystem of claim 6, wherein a weir is formed within the plenum space in each of the upper corners of the frame.

8. A system for purifying bioactive proteins in a fluid, the system comprising:
at least one fractal subsystem, each fractal subsystem comprising:
a resin chamber; and
a pair of fractal packs, wherein the pair of fractal packs is disposed on opposing sides of the resin chamber; and
a filter press structured and arranged to support at least one fractal subsystem in a horizontal arrangement to provide a selectively adjustable bed depth, wherein each fractal subsystem is in fluid communication with every other fractal subsystem.

9. The system of claim 8, wherein the resin chamber comprises:
a frame;
a plenum space through the frame for holding a resin;
a pair of mesh screens for containing the resin within the plenum space; and
a pair of resin ports adapted to at least one of introduce a fluid into the plenum space or remove a fluid from the plenum space.

10. The system of claim 9, wherein the mesh screens comprise a 200 mesh, plain Dutch weave.

11. The system of claim 9, wherein the mesh screens have a nominal opening of 75 microns in the mesh.

12. The system of claim 9, wherein the mesh screens have a geometric opening of 85 microns in the mesh.

13. The system of claim 8, wherein each of the fractal packs comprises:
a frame;
a plenum space through the frame;
a fractal distributor configured within the plenum space;
a fractal flow plate configured within the plenum space; and
a feed material input port adapted to introduce a fluid into the plenum space.

14. The system of claim 13, wherein a weir is formed within the plenum space in each of the upper corners of the frame.

15. The system of claim 8, wherein the filter press comprises:
a pair of vertical supports;
a pair of horizontal supports fixedly attached to the vertical supports, wherein the horizontal supports are structured and arranged to support the fractal packs; and
a filter press subsystem that is structured and arranged to apply a compressive force to the fractal packs.

16. The system of claim 15, wherein the filter press subsystem comprises:
a selectively movable rod;
a follower disposed at a distal end of the movable rod; and
a hydraulic press disposed at a proximal end of the movable rod.

17. The system of claim 8, wherein the selectively adjustable bed depth ranges between one (1) inch and eight (8) feet.

18. The system of claim 8, wherein the system is capable of bidirectional flow.

19. The system of claim 8, wherein the system produces laminar flow in the fluid.

20. The system of claim 8, wherein the fluid comprises a supply feed material with a fat content greater than 0.1 percent and less than five (5) percent.
